(19) United States
(12) Patent Application Publication
(10) Pub. No.: US 2021/0161497 A1
(43) Pub. Date: Jun. 3, 2021

(54) X-RAY IMAGING APPARATUS

(71) Applicant: Shimadzu Corporation, Kyoto (JP)

(72) Inventors: Yuichiro KIRIYAMA, Kyoto (JP); Takuya AOKI, Kyoto (JP); Takatoshi MIWA, Kyoto (JP); Hiroshi OKUMURA, Kyoto (JP)

(21) Appl. No.: 17/105,857

(22) Filed: Nov. 27, 2020

(30) Foreign Application Priority Data
Nov. 29, 2019 (JP) 2019-217170

(51) Int. Cl. A61B 6/00

(52) U.S. Cl. CPC A61B 6/5247; G01N 23/04; A61B 6/547

(57) ABSTRACT

An X-ray imaging apparatus is provided with a front side operation unit provided on a front side of a ceiling-suspended support unit to operate a movement of the ceiling-suspended support unit by a drive unit from a front side of the ceiling-suspended support and a rear side operation unit provided on a rear side of the ceiling-suspended support unit with respect to the front side operation unit and configured to operate the movement of the ceiling-suspended support unit by the drive unit from a rear side of the ceiling-suspended support.

### CROSS-REFERENCE TO RELATED APPLICATIONS

The related application number JP2019-217170, entitled “X-ray imaging apparatus”, filed on Nov. 29, 2019, and invented by Yuichiro Kiriyama, Takuya Aoki, Takatoshi Miwa, and Hiroshi Okumura, upon which this patent application is based, is hereby incorporated by reference.

### BACKGROUND OF THE INVENTION

### Field of the Invention

The present invention relates to an X-ray imaging apparatus.

### Description of the Background Art

Conventionally, an X-ray imaging apparatus equipped with a ceiling-suspended support unit is known. Such an apparatus is disclosed in, for example, Japanese Unexamined Patent Application Publication No. 2010-194152.

Japanese Unexamined Patent Application Publication No. 2010-194152 discloses a radiographic imaging apparatus (X-ray imaging apparatus) equipped with an overhead traveling unit (ceiling-suspended support unit). This overhead traveling unit includes an operation block equipped with an X-ray tube. This operation block is provided with a handle (front side operation unit) to operate the movement of the operation block. In the radiographic imaging apparatus described in Japanese Unexamined Patent Application Publication No. 2010-194152, the operator grips the handle and moves the operation block and stops it at a desired position. In the radiographic imaging apparatus described in Japanese Unexamined Patent Application Publication No. 2010-194152, a handle is provided only on the front side.

However, in the radiographic imaging apparatus described in Japanese Unexamined Patent Application Publication No. 2010-194152, since the handle is provided only on the front side, when the operator is working on the rear side, the operator is required to move the operation block by operating the handle by going around to the front side. Further, when an obstacle, such as, e.g., a patient before imaging, is disposed on the front side with respect to the handle, it is difficult to operate the handle in the first place. Therefore, in the radiographic imaging apparatus described in Japanese Unexamined Patent Application Publication No. 2010-194152, there is a problem that the operational convenience of moving the overhead traveling unit is low.

### SUMMARY OF THE INVENTION

The present invention has been made to solve the above-described problems, and an object of the present invention is to provide an X-ray imaging apparatus capable of improving operational convenience of moving a ceiling-suspended support unit.

In order to achieve the above-described object, an X-ray imaging apparatus according to one aspect of the present invention includes:
an X-ray irradiation unit configured to emit X-rays to a subject;
an X-ray detection unit configured to detect the X-rays emitted from the X-ray irradiation unit;
a ceiling-suspended support unit configured to support the X-ray irradiation unit or the X-ray detection unit;
a drive unit configured to drive and move the ceiling-suspended support unit;
a front side operation unit provided on a front side of the ceiling-suspended support unit and configured to operate a movement of the ceiling-suspended support unit by the drive unit from the front side; and
a rear side operation unit provided on a rear side of the ceiling-suspended support unit with respect to the front side operation unit and configured to operate the movement of the ceiling-suspended support unit by the drive unit from the rear side.

Note that in this specification, the term “movement” is a broad concept including not only a linear movement but also a rotational movement.

According to the present invention, as described above, there are provided: a front side operation unit provided on a front side of the ceiling-suspended support unit and configured to operate a movement of the ceiling-suspended support unit by the drive unit from the front side; and a rear side operation unit provided on a rear side of the ceiling-suspended support unit with respect to the front side operation unit and configured to operate the movement of the ceiling-suspended support unit by the drive unit from the rear side. This allows the operator not only to perform the operation of moving the ceiling-suspended support unit from the front side by the front side operation unit but also to perform the operation of moving the ceiling-suspended support unit from the rear side by the rear side operation unit. As a result, an operator working on the rear side can move the ceiling-suspended support unit by operating the rear side operation unit while staying on the rear side without going around to the front side to operate the front side operation unit. Further, even in cases where an obstacle, such as, e.g., a patient before imaging, is placed on the front side with respect to the front side operation unit, it is possible to move the ceiling-suspended support unit by operating the rear side operation unit. As a result, the operational convenience of moving the ceiling-suspended support unit can be improved.

### BRIEF DESCRIPTION OF THE DRAWINGS

FIG. 1 is a front view showing a configuration of an X-ray imaging apparatus.

FIG. 2 is a block diagram showing a control configuration of the X-ray imaging apparatus.

FIG. 3 is a schematic diagram for explaining a configuration of a ceiling-suspended support unit.

FIG. 4 is a schematic diagram for explaining a configuration of a rear side operation unit.

### DESCRIPTION OF THE PREFERRED EMBODIMENT

(Configuration of X-Ray Imaging Apparatus)

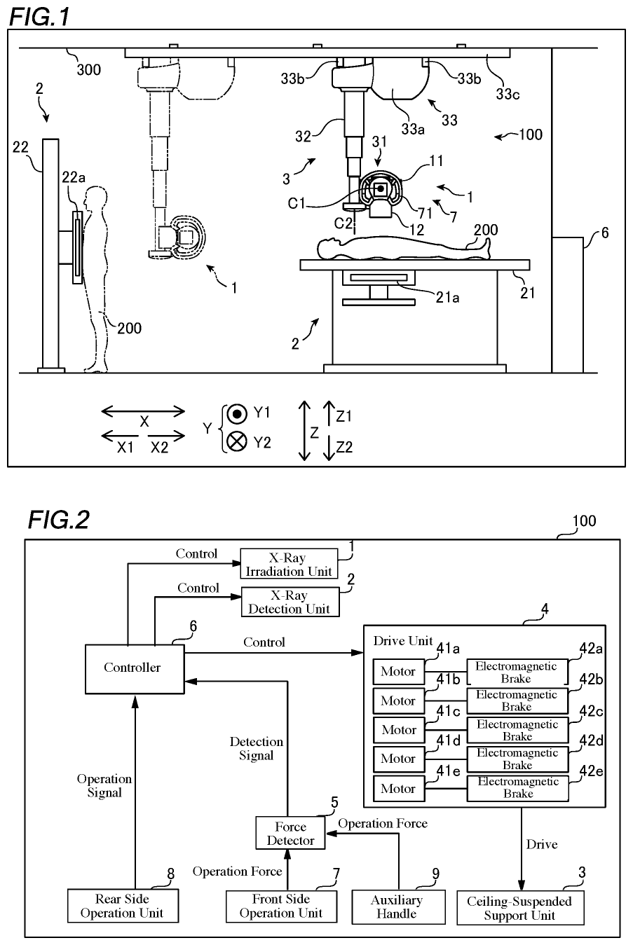

Referring to FIG. 1 to FIG. 4, the entire configuration of an X-ray imaging apparatus 100 according to an embodiment of the present invention will be described.

In the following description, the vertical direction (up-down direction) is defined as a Z-direction, the upward direction is defined as a Z1-direction, the downward direction is defined as a Z2-direction. Further, the front-rear direction (horizontal direction) is defined as a Y-direction, a frontward direction toward the front side is defined as a Y1-direction, and a rearward direction toward the rear side is defined as a Y2-direction. Further, a left-right direction (horizontal direction) perpendicular to the front-rear direction is defined as an X-direction, a left direction when viewed from the front side is defined as an X1-direction, and a right direction when viewed from the front side is defined as an X2-direction.

As shown in FIG. 1 and FIG. 2, the X-ray imaging apparatus 100 according to this embodiment is a medical X-ray imaging apparatus and is configured to perform X-ray imaging of a subject 200 (human body) which is an imaging target. Further, the X-ray imaging apparatus 100 is a generic X-ray imaging apparatus equipped with an overhead traveling type X-ray tube suspension device. The X-ray imaging apparatus 100 is provided with an X-ray irradiation unit 1, an X-ray detection unit 2, a ceiling-suspended support unit 3 (suspension device), a drive unit 4, a force detector 5, a controller 6, a front side operation unit 7, a rear side operation unit 8, and an auxiliary handle 9.

In the X-ray imaging apparatus 100, the X-ray irradiation unit 1 is supported so as to be suspended from the ceiling 300 by the ceiling-suspended support unit 3. The X-ray irradiation unit 1 is movably supported in the imaging chamber by the ceiling-suspended support unit 3. The X-ray irradiation unit 1 includes an X-ray source 11 for emitting X-rays to a subject 200, and a collimator 12 for adjusting the irradiation field of the X-rays.

Further, the X-ray imaging apparatus 100 is provided with an X-ray detection unit 2 for detecting X-rays emitted from the X-ray irradiation unit 1. As shown in FIG. 1, the X-ray detection unit 2 includes a laying posture imaging table 21 for imaging a subject 20 in a posture (laying posture) in which the subject 200 is laid and a standing posture imaging stand 22 for imaging a subject 200 in a posture (standing posture) in which the subject 200 is standing. A laying posture detector 21a and a standing posture detector 22a are movably held depending on the imaging site of the subject 200 on the laying posture imaging table 21 and the standing posture imaging stand 22, respectively. The laying posture detector 21a and the standing posture detector 22a each are, for example, an FPD (Flat Panel Detector) and detect X-rays transmitted through the subject 200.

The ceiling-suspended support unit 3 movably supports the X-ray irradiation unit 1. The ceiling-suspended support unit 3 is configured to be able to move the X-ray irradiation unit 1 from a position where X-ray imaging is performed using the laying posture imaging table 21 to a position where X-ray imaging is performed using the standing posture imaging stand 22. When X-ray imaging is performed using the laying posture imaging table 21, the ceiling-suspended support unit 3 can move the X-ray irradiation unit 1 to a position facing the laying posture detector 21a. Further, the ceiling-suspended support unit 3 can move the X-ray irradiation unit 1 to a position facing the standing posture detector 22a when performing X-ray imaging using the standing posture imaging stand 22.

As shown in FIG. 1 to FIG. 3, the ceiling-suspended support unit 3 includes a rotary support unit 31 for rotatably supporting the X-ray irradiation unit 1 in two directions, i.e., the θ-direction and the φ-direction, the support unit 32 for movably supporting the rotary support unit 31 along one axis (Z-axis) of the vertical direction, and a carriage unit 33 for movably supporting the support unit 32 along two axes (X-axis and Y-axis) of the horizontal direction. That is, the ceiling-suspended support unit 3 is configured to movably support the X-ray irradiation unit 1 in two rotation directions, i.e., the θ-direction and the φ-direction, and in three straight traveling directions, i.e., in the Z-direction, the X-direction, and the Y-direction.

The rotary support unit 31 is attached to the lower portion of the support unit 32. Further, the rotary support unit 31 is configured to rotatably support the X-ray irradiation unit 1 about the first rotation axis C1 extending along the Y-direction as an axis in the θ-direction (the rotation direction about the first rotation axis C1) with respect to the support unit 32. Further, the rotary support unit 31 is configured to rotatably support the X-ray irradiation unit 1 about the second rotation axis C2 extending along the Z-direction as an axis in the φ-direction (the rotation direction about the second rotation axis C2) with respect to the support unit 32.

The support unit 32 is configured to be extendable and retractable in the vertical direction (Z-direction). The rotary support unit 31 is moved in the vertical direction when the support unit 32 is extended and retracted. With this, the X-ray irradiation unit 1 is moved in the vertical direction.

The carriage unit 33 is configured to movably support the support unit 32 in the X-direction and the Y-direction with respect to the ceiling 300. Specifically, the carriage unit 33 includes a support member 33a, a movable rail 33b, and a fixed rail 33c. The support member 33a is configured to support the upper portion of the support unit 32. Further, the support member 33a is mounted so as to be movable in the Y-direction with respect to the movable rail 33b extending in the Y-direction. Further, the movable rail 33b is mounted so as to be movable in the X-direction with respect to the fixed rail 33c extending in the X-direction. The fixed rail 33c is fixed to the ceiling 300. As the movable rail 33b moves on the fixed rail 33c, the X-ray irradiation unit 1, the rotary support unit 31, and the support unit 32 are moved in the X-direction. Further, as the support member 33a moves on the movable rail 33b, the X-ray irradiation unit 1, the rotary support unit 31, and the support unit 32 are moved in the Y-direction.

As shown in FIG. 2, the drive unit 4 is configured to drive and move the ceiling-suspended support unit 3. Specifically, the drive unit 4 includes a plurality of (five in this embodiment) motors 41a to 41e. The motor 41a rotates the rotary support unit 31 in the θ-direction about the first rotation axis C1 as a rotation axis with respect to the support unit 32. The motor 41b rotates the rotary support unit 31 in the φ-direction about the second rotation axis C2 as a rotation axis with respect to the support unit 32. The motor 41c moves the support unit 32 (makes the support unit 32 extend and retract) in the Z-direction. The motor 41d moves the movable rail 33b in the X-direction. The motor 41e moves the support member 33a in the Y-direction. Further, the drive unit 4 includes a plurality of (five in this embodiment) electromagnetic brakes 42a to 42e corresponding to a plurality of (five in this embodiment) motors 41a to 41e.

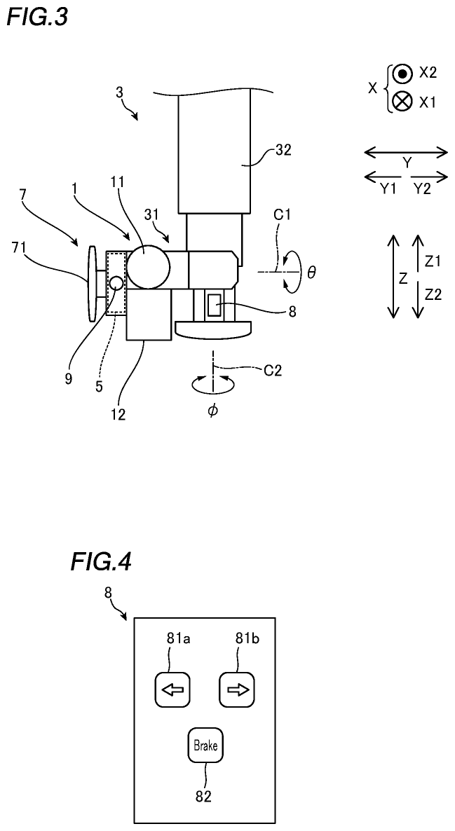

As shown in FIG. 2 and FIG. 3, the force detector 5 is, for example, a strain gauge. The force detector 5 is configured to detect the operation force applied to move the ceiling-suspended support unit 3 by the operator. Specifically, the force detector 5 is configured to commonly detect the operation force inputted (applied) to the main handle 71 and the auxiliary handle 9 of the front side operation unit 7 which will be described later. That is, the force detector 5 is connected to both the main handle 71 and the auxiliary handle 9 of the front side operation unit 7.

The force detector 5 is configured to detect the magnitude and the direction of the input operation force. Further, the force detector 5 is configured to output a detection signal corresponding to the magnitude and the direction of the input operation force. With this, the operator can drive and move the ceiling-suspended support unit 3 in five directions, i.e., the X-direction, the Y-direction, the Z-direction, the θ-direction, and the φ-directions, by gripping and moving the main handle 71 or the auxiliary handle 9 of the front side operation unit 7.

As shown in FIG. 1 and FIG. 2, the controller 6 is a control circuit including a processor, such as, e.g., a CPU (Central Processing Unit), and a memory. The controller 6 is configured to control the operation of each part of the X-ray imaging apparatus 100, such as the X-ray irradiation unit 1, the X-ray detection unit 2, and the drive unit 4. The controller 6 is configured to control the common drive unit 4 to drive and move the ceiling-suspended support unit 3 based on the operation of the front side operation unit 7, the operation of the rear side operation unit 8, and the operation of the auxiliary handle 9.

As shown in FIG. 1 to FIG. 3, the front side operation unit 7 is provided on the front side (Y1-direction side) of the ceiling-suspended support unit 3 to operate the movement of the ceiling-suspended support unit 3 by the drive unit 4 from the front side. The front side operation unit 7 is a handle operation unit including a main handle 71. The main handle 71 is configured to be gripped by an operator when moving the ceiling-suspended support unit 3 from the front side. Specifically, the main handle 71 is configured to be gripped when an operator performs an operation of manually moving the ceiling-suspended support unit 3 from the front side. The main handle 71 has a wheel-shape and is configured to be gripped by an operator with both hands. The main handle 71 of the front side operation unit 7 is arranged on the front side (Y1-direction side) with respect to the force detector 5 and is connected to the force detector 5 from the front side. Note that the main handle 71 is an example of the “handle” recited in claims.

Here, in this embodiment, as shown in FIG. 2 to FIG. 4, the rear side operation unit 8 is provided on the rear side (Y2-direction side) of the ceiling-suspended support unit 3 with respect to the front side operation unit 7 to operate the movement of the ceiling-suspended support unit 3 by the drive unit 4 from the rear side. Specifically, the rear side operation unit 8 is provided on the rear side than the center of the ceiling-suspended support unit 3 in the Y-direction. The rear side operation unit 8 is a switch operation unit including a plurality of switches 81a, 81b, and 82. Specifically, the rear side operation unit 8 is a seat-type switch operation unit. For this reason, the front side operation unit 7 and the rear side operation unit 8 are operation units different in type. The rear side operation unit 8 is provided on the side surface (X2-direction side surface) of the rotary support unit 31 of the ceiling-suspended support unit 3.

The switches 81a and 81b are each configured to receive an instruction of a moving direction of the ceiling-suspended support unit 3. Specifically, the switch 81a and 81b are each configured to receive an instruction to move the ceiling-suspended support unit 3 in the X-direction. More specifically, the switch 81a is configured to receive an instruction to move the ceiling-suspended support unit 3 in the X1-direction. Further, the switch 81b is configured to receive an instruction to move the ceiling-suspended support unit 3 in the X2-direction. Further, the switch 82 is configured to receive an instruction to release the braking of the motors 41a to 41e by the electromagnetic brakes 42a to 42e. The switch 81a, 81b, and 82 each are a push-button switch. The rear side operation unit 8 is configured to output an operation signal corresponding to the pressing operation of the switch 81a, 81b, or 82.

The rear side operation unit 8 is configured to receive an instruction to drive the ceiling-suspended support unit 3 in a particular moving direction (the X-direction in this embodiment) among a plurality of moving directions (five directions in this embodiment, i.e., the X-direction, the Y-direction, the Z-direction, the θ-direction, and the φ-direction) of the ceiling-suspended support unit 3. For this reason, the number (one in this embodiment) of moving directions of the ceiling-suspended support unit 3 operable by the rear side operation unit 8 is less than the number (five in this embodiment) of moving directions of the ceiling-suspended support unit 3 operable by the front side operation unit 7. The moving directions of the ceiling-suspended support unit 3 operable by the front side operation unit 7 include the up-down direction (the Z-direction), the horizontal directions (the X-direction and the Y-direction), and the rotation directions (the θ-direction and the φ-direction). Further, the moving direction of the ceiling-suspended support unit 3 operable by the rear side operation unit 8 includes a horizontal direction (the X-direction).

In this embodiment, as shown in FIG. 2 and FIG. 3, the auxiliary handle 9 is independently provided separately from the rear side operation unit 8 on the rear side (Y2-direction side) with respect to the main handle 71 of the front side operation unit 7 to operate the movement of the ceiling-suspended support unit 3 by the drive unit 4 from the rear side. Specifically, the auxiliary handle 9 is provided on the rear side with respect to the main handle 71 of the front side operation unit 7 and is provided at a position on the side surface side (X2-direction side) with respect to the force detector 5. The auxiliary handle 9 is arranged on the side surface side with respect to the force detector 5 and is connected to the force detector 5 from the side surface side. The auxiliary handle 9 is provided on the side surface of the ceiling-suspended support unit 3 on the X2-direction side. Note that the auxiliary handle 9 is not provided on the side surface of the ceiling-suspended support unit 3 in the X1-direction. The auxiliary handle 9 is provided in the vicinity of the front side operation unit 7.

The auxiliary handle 9 is configured to be gripped by an operator when moving the ceiling-suspended support unit 3 from the rear side. Specifically, the auxiliary handle 9 is configured to be gripped when the operator manually moves the ceiling-suspended support unit 3 from the rear side. The auxiliary handle 9 has a knob shape protruding in the X2-direction and is configured to be gripped by an operator with one hand. The auxiliary handle 9 is smaller than the main handle 71 of the front side operation unit 7. The auxiliary handle 9 is smaller than the main handle 71 of the front side operation unit 7 at least in the Z-direction. Also, the auxiliary handle 9 may be smaller than the main handle 71 of the front side operation unit 7 in the X-direction and the Y-direction. The auxiliary handle 9 is formed to be smaller than the main handle 71 so as not to interfere with the main handle 71 of the front side operation unit 7.

(Operation to Move Ceiling-suspended Support Unit)

Next, the movements of the ceiling-suspended support unit 3 using the front side operation unit 7, the rear side operation unit 8, and the auxiliary handle 9 will be described in order.

For example, when an operator is working on the front side, the operator performs the operation to move the ceiling-suspended support unit 3 by gripping and moving the main handle 71 on the front side operation unit 7. In this case, the force detector 5 detects the magnitude and the direction of the operation force applied to the main handle 71 of the front side operation unit 7 by the operator. The controller 6 controls the drive unit 4 so as to drive and move the ceiling-suspended support unit 3 in accordance with the magnitude and the direction of the operation force detected by the force detector 5. Consequently, the drive unit 4 drives and moves the ceiling-suspended support unit 3 in a direction corresponding to the direction of the operation force applied to the main handle 71 of the front side operation unit 7 at a speed corresponding to the magnitude of the operation force applied to the main handle 71 of the front side operation unit 7. That is, the power-assist operation for the operator's operation force is performed. With this, the operator can move the ceiling-suspended support unit 3 to the desired position using the main handle 71 of the front side operation unit 7.

Further, for example, when an operator is working on the rear side, the operator performs the operation to move the ceiling-suspended support unit 3 using the rear side operation unit 8 or the auxiliary handle 9.

For example, when it is desired to move the ceiling-suspended support unit 3 in the X-direction, the operator uses the rear side operation unit 8. When using the rear side operation unit 8, the operator performs the operation to move the ceiling-suspended support unit 3 by depressing the switch 81a or 81b of the rear side operation unit 8. In this case, the controller 6 controls the drive unit 4 so as to move the ceiling-suspended support unit 3 in response to the depressing operation of the switch 81a or 81b of the rear side operation unit 8. For example, the controller 6 controls the drive unit 4 so as to drive and move the ceiling-suspended support unit 3 in the X1-direction when the switch 81a of the rear side operation unit 8 is depressed.

Further, for example, the controller 6 controls the drive unit 4 so as to drive and move the ceiling-suspended support unit 3 in the X2-direction when the switch 81b of the rear side operation unit 8 is depressed. The drive unit 4 drives and moves the ceiling-suspended support unit 3 at a constant rate in a direction corresponding to the switch 81a or 81b of the rear side operation unit 8. That is, not a power assist operation but a normal movement operation at a predetermined speed is performed. With this, the operator can move the ceiling-suspended support unit 3 to a desired position in the X-direction using the switches 81a and 81b of the rear side operation unit 8.

Note that the controller 6 controls the drive unit 4 so as to move and drive the ceiling-suspended support unit 3 while the switch 81a or 81b of the rear side operation unit 8 is being depressed. That is, only while the switch 81a or 81b of the rear side operation unit 8 is being depressed, the ceiling-suspended support unit 3 is driven and moved by the drive unit 4.

Further, for example, when it is desired to move the ceiling-suspended support unit 3 in a direction other than the X-direction, the operator uses the auxiliary handle 9. When using the auxiliary handle 9, the operator performs the operation to move the ceiling-suspended support unit 3 by gripping and moving the auxiliary handle 9 while depressing the switch 82 of the rear side operation unit 8. In other words, the operator performs the operation to move the ceiling-suspended support unit 3 by gripping and moving the auxiliary handle 9 in a state in which the braking of the motor 41a to 41e by the electromagnetic brake 42a to 42e is released.

Note that in this embodiment, since the auxiliary handle 9 and the switch 82 are provided so as to be operable from the same side (on the X2-direction-side), it is easy to simultaneously operate the auxiliary handle 9 and the switch 82.

In this case, the movement of the ceiling-suspended support unit 3 is the same as that of the main handle 71 of the front side operation unit 7. That is, the force detector 5 detects the magnitude and the direction of the operation force applied to the auxiliary handle 9 by the operator. The controller 6 controls the drive unit 4 so as to drive and move the ceiling-suspended support unit 3 in accordance with the magnitude and the direction of the operation force detected by force detector 5. As a result, the drive unit 4 drives and moves the ceiling-suspended support unit 3 in a direction corresponding to the direction of the operation force applied to the auxiliary handle 9 at a speed corresponding to the magnitude of the operation force applied to the auxiliary handle 9. That is, even when the auxiliary handle 9 is used, the power-assist operation is performed for the operator's operation force. With this, the operator can move the ceiling-suspended support unit 3 to the desired position by using the auxiliary handle 9.

### Effects of Embodiment

In this embodiment, the following effects can be obtained.

In this embodiment, as described above, the X-ray imaging apparatus 100 is provided with the front side operation unit 7 provided on the front side of the ceiling-suspended support unit 3 to operate the movement of the ceiling-suspended support unit 3 by the drive unit 4 from the front side and the rear side operation unit 8 provided on the rear side of the ceiling-suspended support unit 3 with respect to the front side operation unit 7 to operate the movement of the ceiling-suspended support unit 3 by the drive unit 4 from the rear side.

With this, the operator not only can perform the operation to move the ceiling-suspended support unit 3 from the front side by the front side operation unit 7 but also can perform the operation to move the ceiling-suspended support unit 3 from the rear side by the rear side operation unit 8. With this, the operator working on the rear side can move the ceiling-suspended support unit 3 by operating the rear side operation unit 8 while staying on the rear side without operating the front side operation unit 7 by going around to the front side.

Further, even in cases where an obstacle, such as, e.g., a patient before imaging, is disposed on the front side with respect to the front side operation unit 7, the ceiling-suspended support unit 3 can be moved by operating the rear side operation unit 8. As a result, the operational convenience of moving the ceiling-suspended support unit 3 can be improved.

Further, in this embodiment, as described above, the front side operation unit 7 and the rear side operation unit 8 are configured to be operation units different in type.

With this, unlike the case in which the front side operation unit 7 and the rear side operation unit 8 are configured to be operation units of the same type, between the front side operation unit 7 and the rear side operation unit 8, the rear side operation unit 8 can be a simple structure with respect to the front side operation unit 7. As a result, even in cases where both the front side operation unit 7 and the rear side operation unit 8 are provided, it is possible to suppress the complexity of the structure of the X-ray imaging apparatus 100.

Further, in this embodiment, as described above, the front side operation unit 7 is configured to be a handle operation unit including the main handle 71 (handle).

Further, the rear side operation unit 8 is configured to be a switch operation unit including the switches 81a, 81b, and 82. With this, unlike the case in which the rear side operation unit 8 is configured to be the same handle operation unit as that of the front side operation unit 7, there is no need to provide a sensor such as the force detector 5 to the rear side operation unit 8, so that between the front side operation unit 7 and the rear side operation unit 8, the rear side operation unit 8 can be more simplified as compared with the front side operation unit 7. As a result, even in cases where both the front side operation unit 7 and the rear side operation unit 8 are provided, it is possible to further suppress the complexity of the structure of the X-ray imaging apparatus 100.

Further, in this embodiment, as described above, the X-ray imaging apparatus 100 is configured such that the number of moving directions of the ceiling-suspended support unit 3 operable by the rear side operation unit 8 is smaller than the number of moving directions of the ceiling-suspended support unit 3 operable by the front side operation unit 7.

With this, as compared with the case in which the X-ray imaging apparatus 100 is configured such that the number of moving directions of the ceiling-suspended support unit 3 operable by the rear side operation unit 8 is the same as the number of moving directions of the ceiling-suspended support unit 3 operable by the front side operation unit 7, the rear side operation unit 8 can be more assuredly made to be simple in structure with respect to the front side operation unit 7 by the smaller number of moving directions.

Further, in this embodiment, as described above, the X-ray imaging apparatus 100 is configured such that the moving direction of the ceiling-suspended support unit 3 operable by the front side operation unit 7 includes the horizontal direction, the up-down direction, and the rotation direction. Further, the X-ray imaging apparatus 100 is configured such that the moving direction of the ceiling-suspended support unit 3 operable by the rear side operation unit 8 includes the horizontal direction.

With this, since the ceiling-suspended support unit 3 can be moved and driven by the front side operation unit 7 in the horizontal direction, the up-down direction, and the rotation direction, when operating the movement of the ceiling-suspended support unit 3 from the front side, the ceiling-suspended support unit 3 can be driven and moved sufficiently in the desired direction. Further, it is possible to drive and move the ceiling-suspended support unit 3 in the horizontal direction which is a direction often desired to be moved when operating the movement of the ceiling-suspended support unit 3 from the rear side by the rear side operation unit 8, even when operating the movement of the ceiling-suspended support unit 3 from the rear side, the ceiling-suspended support unit 3 can be driven and moved sufficiently in a desired direction.

Further, in this embodiment, as described above, the front side operation unit 7 is configured to include the main handle 71. Further, the X-ray imaging apparatus 100 is configured to include the auxiliary handle 9 independently provided separately from the rear side operation unit 8 on the rear side with respect to the main handle 71 to operate the movement of the ceiling-suspended support unit 3 by the drive unit 4 from the rear side.

With this, the movement operation of the ceiling-suspended support unit 3 can be performed from the rear side using the auxiliary handle 9 positioned on the rear side with respect to the main handle 71 even if the operator's hand cannot reach the main handle 71 from the rear side when the handle operation is desired from the rear side. As a result, the operational convenience of moving the ceiling-suspended support unit 3 can be further improved.

Further, in this embodiment, as described above, the auxiliary handle 9 is configured to be smaller than the main handle 71. With this, even in the case of providing the auxiliary handle 9, it is possible to easily suppress the enlargement of the structure of the X-ray imaging apparatus 100.

Further, in this embodiment, as described above, the X-ray imaging apparatus 100 is configured to include the force detector 5 to commonly detect the operation force applied to the main handle 71 and the operation force applied to the auxiliary handle 9.

With this, since the force detector 5 can be shared by the main handle 71 and the auxiliary handle 9, the number of components can be reduced and the structure can be simplified as compared with the case in which the force detector 5 is separately provided for each of the main handle 71 and the auxiliary handle 9.

Further, in this embodiment, as described above, the X-ray imaging apparatus 100 is configured to include the controller 6 for controlling the common drive unit 4 so as to drive and move the ceiling-suspended support unit 3 based on the operation of the front side operation unit 7 and the operation of the rear side operation unit 8.

With this, since the drive unit 4 and the controller 6 can be shared by the front side operation unit 7 and the rear side operation unit 8, the number of components can be reduced and the structure can be simplified as compared with the case in which the drive unit 4 and the controller 6 are separately provided for each of the front side operation unit 7 and the rear side operation unit 8.

### Modifications

It should be understood that the embodiments disclosed here are examples in all respects and are not restrictive. The scope of the present invention is indicated by the appended claims rather than by the description of the above-described embodiments and includes all modifications (changes) within the meanings and the scopes equivalent to the scope of the claims.

For example, in the above embodiment, an example is shown in which the rear side operation unit is a switch operation unit, but the present invention is not limited thereto. In the present invention, the rear side operation unit may be a handle operation unit. That is, the front side operation unit and the rear side operation unit may be an operation unit of the same type. Further, when the rear side operation unit is a handle operation unit, it may be provided with a force detector for the rear side operation unit separately from the front side operation unit or may be provided with a common force detector for the front side operation unit and the rear side operation unit. The rear side operation unit may be a stick operation unit including a joystick that accepts a moving direction instruction of the ceiling-suspended support unit.

Further, in the above-described embodiment, the number of moving directions of the ceiling-suspended support unit operable by the rear side operation unit is smaller than the number of moving directions of the ceiling-suspended support unit operable by the front side operation unit, but the present invention is not limited thereto. In the present invention, the number of moving directions of the ceiling-suspended support unit operable by the rear side operation unit may be the same as the number of moving directions of the ceiling-suspended support unit operable by the front side operation unit. That is, the rear side operation unit may be configured to receive the same number of instructions of moving directions of the ceiling-suspended support unit as that of the front side operation unit.

In the above-described embodiment, an example is shown in which the rear side operation unit is configured to receive an instruction to drive in the X-direction, but the present invention is not limited thereto. In the present invention, between the plurality of moving directions (in the above-described embodiment, five directions, i.e., the X-direction, the Y-direction, the Z-direction, the θ-direction, and the φ-direction) of the ceiling-suspended support unit, the rear side operation unit may be configured to receive an instruction of driving in all the moving directions, or may be configured to receive a particular plurality of moving directions (e.g., in the X-direction, the Y-direction, and the Z-direction).

Further, in the above-described embodiment, an example is shown in which the X-ray imaging apparatus is independently provided with an auxiliary handle separately from the rear side operation unit, but the present invention is not limited thereto. In the present invention, it is not always required that the X-ray imaging apparatus is provided with an auxiliary handle.

In the above-described embodiment, an example is shown in which the auxiliary handle has a knob shape protruding in the X2-direction, but the present invention is not limited thereto. In the present invention, the auxiliary handle may have a shape other than a knob shape, such as, e.g., a bar shape and a wheel shape.

Further, in the above-described embodiment, an example is shown in which the X-ray imaging apparatus is provided with a force detector to commonly detect the operation force applied to the main handle and the operation force applied to the auxiliary handle, the present invention is not limited thereto. In the present invention, the X-ray imaging apparatus may be separately and independently provided with a force detector to detect the operation force applied to the main handle and a force detector to detect the operation force applied to the auxiliary handle.

In the above-described embodiment, an example is shown in which the ceiling-suspended support unit is configured to support the X-ray irradiation unit, but the present invention is not limited thereto. In the present invention, the ceiling-suspended support unit may be configured to support the X-ray detection unit.

[Aspects]

It should be understood by those skilled in the art that the above-described exemplary embodiments are concrete examples of the following aspects.

(Item 1)

An X-ray imaging apparatus comprising:
an X-ray irradiation unit configured to emit X-rays to a subject;
an X-ray detection unit configured to detect the X-rays emitted from the X-ray irradiation unit;
a ceiling-suspended support unit configured to support the X-ray irradiation unit or the X-ray detection unit;
a drive unit configured to drive and move the ceiling-suspended support unit;
a front side operation unit provided on a front side of the ceiling-suspended support unit and configured to operate a movement of the ceiling-suspended support unit by the drive unit from the front side; and
a rear side operation unit provided on a rear side of the ceiling-suspended support unit with respect to the front side operation unit and configured to operate the movement of the ceiling-suspended support unit by the drive unit from the rear side.

(Item 2)

The X-ray imaging apparatus as recited in the aforementioned Item 1,
wherein the front side operation unit and the rear side operation unit are operation units different in type.

(Item 3)

The X-ray imaging apparatus as recited in the aforementioned Item 2,
wherein the front side operation unit is a handle operation unit including a handle, and
wherein the rear side operation unit is a switch operation unit including a switch.

(Item 4)

The X-ray imaging apparatus as recited in the aforementioned Item 2 or 3,
wherein the number of moving directions of the ceiling-suspended support unit operable by the rear side operation unit is less than the number of moving directions of the ceiling-suspended support unit operable by the front side operation unit.

(Item 5)

The X-ray imaging apparatus as recited in the aforementioned Item 4,
wherein the moving direction of the ceiling-suspended support unit operable by the front side operation unit includes a horizontal direction, an up-down direction, and a rotation direction, and
wherein a moving direction of the ceiling-suspended support unit operable by the rear side operation unit includes a horizontal direction.

(Item 6)

The X-ray imaging apparatus as recited in any one of the aforementioned Items 1 to 5,
wherein a front side operation unit includes a main handle, and
wherein the X-ray imaging apparatus further comprises an auxiliary handle configured to operate the movement of the ceiling-suspended support unit by the drive unit from the rear side, the auxiliary handle being independently provided separately from the rear side operation unit on the rear side with respect to the main handle.

(Item 7)

The X-ray imaging apparatus as recited in the aforementioned Item 6,
wherein the auxiliary handle is smaller than the main handle.

(Item 8)

The X-ray imaging apparatus as recited in the aforementioned Item 6 or 7, further comprising:
a force detector configured to commonly detect an operation force input to the main handle and an operation force input to the auxiliary handle.

(Item 9)

The X-ray imaging apparatus as recited in any one of the aforementioned Items 1 to 8, further comprising:
a controller configured to control the drive unit commonly used to drive and move the ceiling-suspended support unit based on an operation of the front side operation unit and an operation of the rear side operation unit.

1. An X-ray imaging apparatus comprising:
an X-ray irradiation unit configured to emit X-rays to a subject;
an X-ray detection unit configured to detect the X-rays emitted from the X-ray irradiation unit;
a ceiling-suspended support unit configured to support the X-ray irradiation unit or the X-ray detection unit;
a drive unit configured to drive and move the ceiling-suspended support unit;
a front side operation unit provided on a front side of the ceiling-suspended support unit and configured to operate a movement of the ceiling-suspended support unit by the drive unit from the front side; and
a rear side operation unit provided on a rear side of the ceiling-suspended support unit with respect to the front side operation unit and configured to operate the movement of the ceiling-suspended support unit by the drive unit from the rear side.

2. The X-ray imaging apparatus as recited in claim 1,
wherein the front side operation unit and the rear side operation unit are operation units different in type.

3. The X-ray imaging apparatus as recited in claim 2,
wherein the front side operation unit is a handle operation unit including a handle, and
wherein the rear side operation unit is a switch operation unit including a switch.

4. The X-ray imaging apparatus as recited in claim 2,
wherein the number of moving directions of the ceiling-suspended support unit operable by the rear side operation unit is less than the number of moving directions of the ceiling-suspended support unit operable by the front side operation unit.

5. The X-ray imaging apparatus as recited in claim 4,
wherein the moving direction of the ceiling-suspended support unit operable by the front side operation unit includes a horizontal direction, an up-down direction, and a rotation direction, and
wherein a moving direction of the ceiling-suspended support unit operable by the rear side operation unit includes a horizontal direction.

6. The X-ray imaging apparatus as recited in claim 1,
wherein a front side operation unit includes a main handle, and
wherein the X-ray imaging apparatus further comprises an auxiliary handle configured to operate the movement of the ceiling-suspended support unit by the drive unit from the rear side, the auxiliary handle being independently provided separately from the rear side operation unit on the rear side with respect to the main handle.

7. The X-ray imaging apparatus as recited in claim 6,
wherein the auxiliary handle is smaller than the main handle.

8. The X-ray imaging apparatus as recited in claim 6, further comprising:
a force detector configured to commonly detect an operation force input to the main handle and an operation force input to the auxiliary handle.

9. The X-ray imaging apparatus as recited in claim 1, further comprising:
a controller configured to control the drive unit commonly used to drive and move the ceiling-suspended support unit based on an operation of the front side operation unit and an operation of the rear side operation unit.
